(19) United States
(12) Patent Application Publication
(10) Pub. No.: US 2019/0374374 A1
(43) Pub. Date: Dec. 12, 2019

(54) SYSTEMS AND METHODS FOR REDUCING SNORING AND/OR SLEEP APNEA

(71) Applicant: SNOREX LLC, SAINT PAUL, MN (US)

(72) Inventors: ADAM BLACK, PLYMOUTH, MN (US); BRIAN KROHN, SAINT PAUL, MN (US); Umesh Goswami, NORTH OAKS, MN (US)

(73) Assignee: SNOREX LLC

(21) Appl. No.: 16/547,593

(22) Filed: Aug. 22, 2019

Related U.S. Application Data
(63) Continuation of application No. PCT/US2018/019171, filed on Feb. 22, 2018.
(60) Provisional application No. 62/462,033, filed on Feb. 22, 2017.

(51) Int. Cl. A61F 5/56; A61M 21/00

(52) U.S. Cl. CPC A61F 5/56; A61M 21/00; A61B 5/4818; A61M 2205/3375; A61M 2205/583; A61M 2021/005

(57) ABSTRACT

A graphical user interface is used to provide training to conduct vocal exercises and provide feedback in order to reduce snoring and/or obstructive sleep apnea. The vocal exercises are associated with, for example, strength, coordination, and endurance for muscles and nerve control associated with sleep apnea or snoring. This summary is not intended to describe each embodiment or every implementation of the subject matter hereof. The figures and the detailed description more particularly exemplify various embodiments.

### CROSS-REFERENCE TO RELATED APPLICATIONS

This application is a continuation of International Patent Application No. PCT/US2018/019171 with a filing date of Feb. 22, 2018, designating the United States, now pending, and further claims priority to U.S. Provisional Patent Application No. 62/462,033, filed on 22 Feb. 2017. The content of the aforementioned applications, including any intervening amendments thereto, are incorporated herein by reference.

### TECHNICAL FIELD

Embodiments include methods and systems to promote stimulation of the hypoglossal nerve and the genioglossus muscle to reduce or prevent snoring, sleep apnea and/or breathing obstructions caused by inadequate motor tone/neuromuscular tone of the tongue and/or pharyngeal wall muscles.

### BACKGROUND

Obstructive sleep apnea (OSA) is a disease characterized by the relaxation of the throat muscles that block the airway during sleep. OSA affects 1 in 5 Americans and can lead to many serious health conditions such as hypertension, stroke, heart disease, arrhythmias, diabetes, and depression. OSA has also been shown increase the risk of cancer mortality by 4.8 times by promoting angiogenesis in tumors. In addition to the serious health implications of OSA, OSA also has considerable economic costs. Estimates of health care costs related to untreated OSA are approximately $4.9 billion in the United States.

Although current solutions for treating OSA are effective, their efficacy is compromised due to low patient compliance. The most common treatment for moderate to severe OSA is the use of a continuous positive airway pressure (CPAP) machine, which forces air into the patient's airway to keep it open. A CPAP machine requires the patient to sleep with a mask over their nose and mouth. This method is not well-tolerated by patients which results in 29 to 83 percent of patients being non-compliant. Alternative options to CPAP for moderate to severe OSA include surgery that removes tissue or inserts stiffening material into the upper airway, or neuro-stimulation that stimulates the hypoglossal nerve, opening the airway. Thus, for moderate to severe sufferers of OSA many of the options are very uncomfortable or invasive.

For mild to moderate OSA, there are a host of less invasive devices, yet patient compliance remains and issue. These treatments include dental appliances that pull the lower jaw forward, chin straps that hold the mouth closed, or sleep position devices that attempt to keep the patient sleep on their side. As with CPAP, long term compliance with these devices is low and a large portion of patients therefore suffer the health repercussions of untreated OSA. Snoring is not technically OSA, because snoring is not a medically diagnosable disease, but it is closely related to OSA and caused by weak upper airway muscles. Most patients with mild to moderate OSA initially seek treatment to address snoring, and many of the treatments for mild to moderate OSA are also applied to snoring but are not reimbursable.

Current therapies for treating obstructive sleep apnea (OSA) are devices that are bulky, uncomfortable and not well tolerated. Alternative solutions include surgeries that are invasive and costly, and for which not all patients are eligible. There is considerable need for a non-invasive and non-wearable alternative to traditional therapies. The current standard of care for moderate to severe OSA is the CPAP machine, which patients greatly dislike because they require the patient to wear a bulky and uncomfortable mask that is connected by hoses to a noisy pump, all of which interfere with the patient's sleep quality. As a result, many moderate to severe OSA patients give up after a few weeks of therapy and their OSA remains untreated. Clinicians are similarly frustrated by the low compliance rates because of the significant health risks posed by even mild to moderate OSA. Furthermore, CPAP is only prescribed to moderate to severe OSA patients, leaving clinicians with limited effective options to offer their mild to moderate patients. Finally, from a payer's perspective, reducing untreated OSA has clear cost savings because of the high health care costs resulting from untreated OSA.

### SUMMARY

A graphical user interface is used to provide training, feedback, and tracking to conduct vocal exercises. The vocal exercises are associated with, for example, strength, coordination, and endurance for muscles and nerve control associated with sleep apnea or snoring.

This summary is not intended to describe each embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1A is a flowchart of a method for conducting exercises to reduce snoring and/or sleep apnea according to an embodiment.

FIG. 1B is a system diagram of a device for treatment of snoring or sleep apnea in a patient according to an embodiment.

FIGS. 2A and 2B are ultrasound views of an upper respiratory system.

FIGS. 3A-3C depict a smartphone application (“app”) for conducting a voice-controlled game to reduce or prevent sleep apnea and/or snoring.

FIGS. 4A-4C depict an alternative embodiment of a smartphone app, including start screens, for conducting a voice-controlled game to reduce or prevent sleep apnea and/or snoring.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

According to embodiments described herein, the challenge of low patient compliance is addressed using OSA therapy and a diagnostic tool. Backed by evidence-based sleep medicine research, a training program can be implemented on, for example, a smartphone application, or “app,” that uses engaging voice-controlled games to build muscle coordination and endurance of the upper airway. Adequate muscle training can reduce apneic event during sleep without the need for bulky devices or invasive procedures. This makes training an attractive option for patients having mild to moderate OSA or who cannot tolerate current therapies. Such a solution could help the 50 million Americans with mild OSA and the 9.3 million adults with moderate OSA who are non-compliant to CPAP therapy and would thus be better served with a non-wearable therapy.

Using ultrasound, the tongue and upper airway can be imaged during speech exercises to validate the hypothesized mechanism of action. With these data a variety of voice-controlled games and exercised can be developed that can be incorporated into a clinical version of a software program such as a smartphone app. Other devices, such as tablets, laptops, computers, and mobile computing devices, also can host the app or other software for implementing and presenting the training program.

A method for exercising the upper airway using specific vocalizations and monitoring those vocalizations can use speech recognition to provide feedback. The feedback can be an interactive game to improve engagement and compliance to the therapy as well as information about how well the patient is doing with the therapy or how well the patient is progressing in his or her therapy.

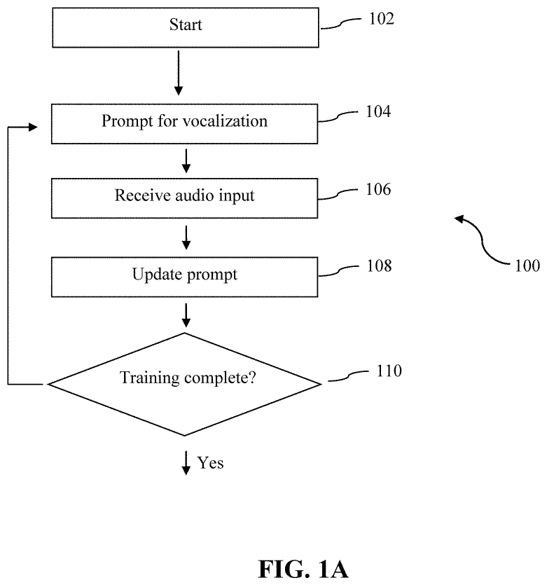

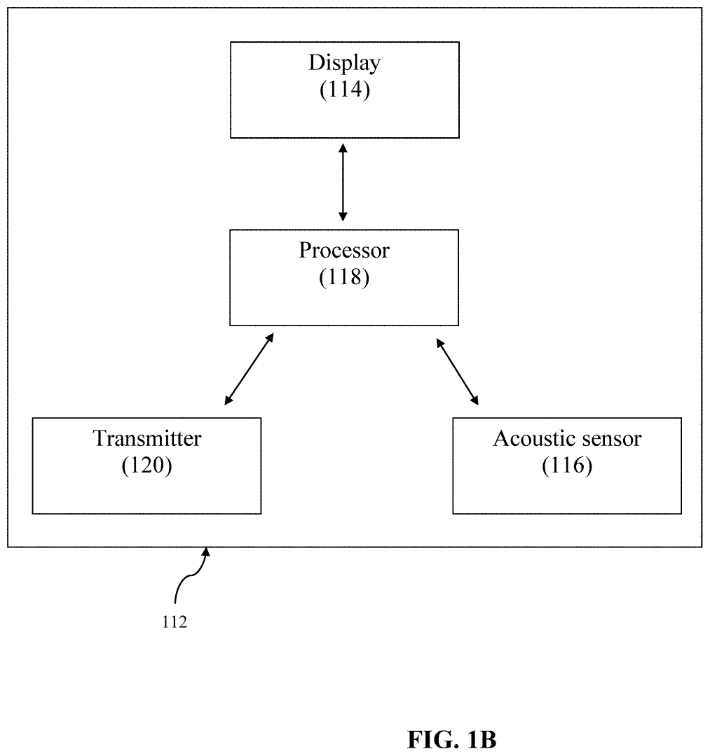

FIG. 1A is a flowchart of one such method 100 for exercising the upper airway using specific vocalizations. Method 100 starts at 102, and subsequently a prompt is generated for a specific vocalization at 104.

Prompt 104 can be an audio, visual, haptic, or any other type of prompt. For example, in one embodiment described below, the prompt can be depicted visually. Visual prompts can take various forms, and can indicate volume, pitch, and type of vocalization, in various embodiments.

At 106, audio input is received. In embodiments, the audio input can be received by a microphone or other audio sensor of or coupled with the same device, such as a smartphone, tablet, laptop, computer, or mobile computing device, that generates the prompt at 104. In alternative embodiments, such as in a clinic, a separate device can receive audio input.

At 108, the prompt is updated. For example, in embodiments in which the prompt is displayed visually at 104, the visual display can be updated based on the audio input at 104. The prompt can be updated to move an indicator, change a color, or otherwise indicate to a user some information regarding the audio input of the vocalization that was received at 104.

At 110, a determination is made of whether a training course is complete. In embodiments, a training program is considered complete when a sufficient number of vocal exercises has been completed, corresponding to a prescribed regimen by a physician, speech therapist or other professional. In some embodiments, the determination of whether the training is complete can be made based on several factors, including the number of vocalizations, the quality of the vocalizations measured at 106, and a preset target for a quantity and type of vocal exercises.

In embodiments, the specific vocalizations are the /i/ and /a/ vowel sounds, which exercise the full range of motion of tongue base, i.e. all the way forward and back. These sounds cannot be produced in any other way—that is, it is not possible for the user to cheat and do the exercises improperly while still generating the requested sound. In other embodiments, the consonant /n/ in the word “knee” can further be used to exercise the soft palate. Various other sounds corresponding to particular muscles and nerve pathways, not all listed here, are contemplated and would be understood by one of ordinary skill in the art. According to some embodiments, the exercises can be used to improve any or all of strengthening, tone, endurance and muscular control (coordination of the brain, nerve, and muscle pathway).

Additionally, some methods may provide user feedback to the user to let them know if they are doing the exercises correctly. For example, in embodiments method 100 may include providing a score corresponding to how close the received audio signal is to the prompted word or sound. Method 100 could provide feedback to indicate if the user is saying less effective but similar sounding words to those which are prompted. In still further embodiments, a mechanical device could be used to register when the tongue is in the correct position.

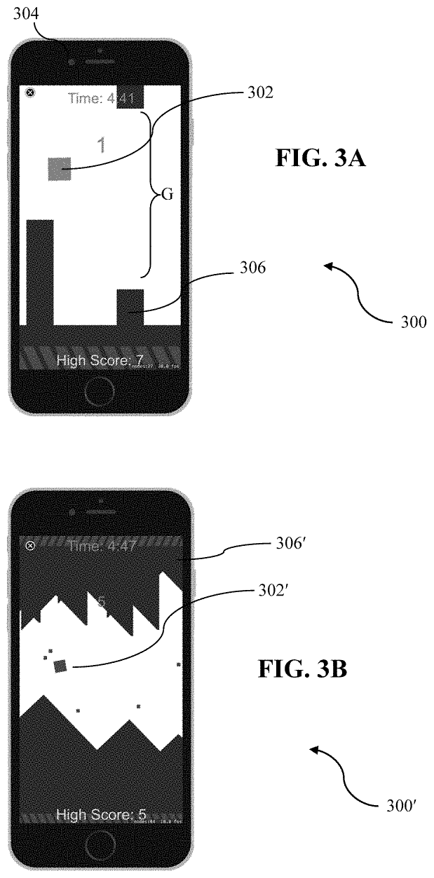

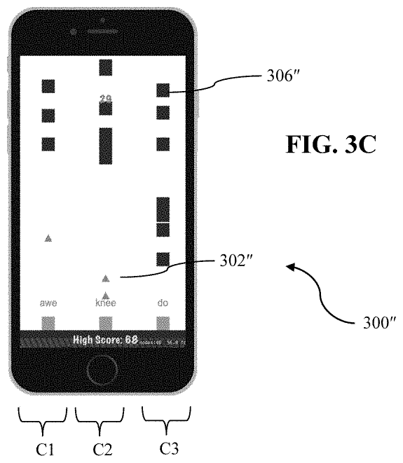

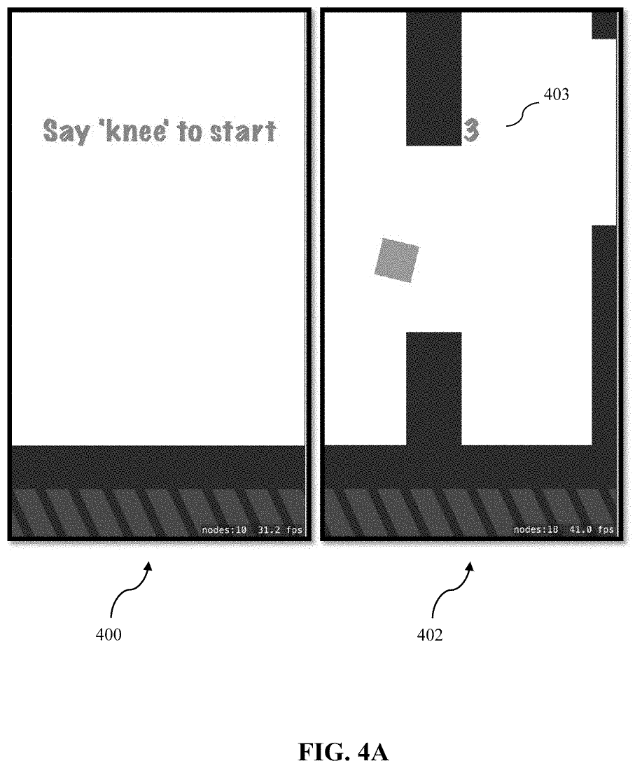

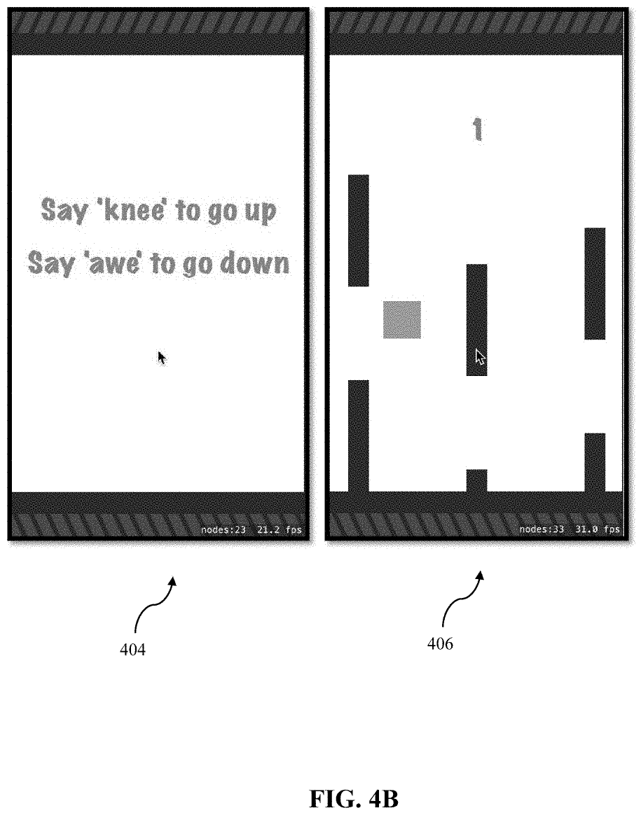

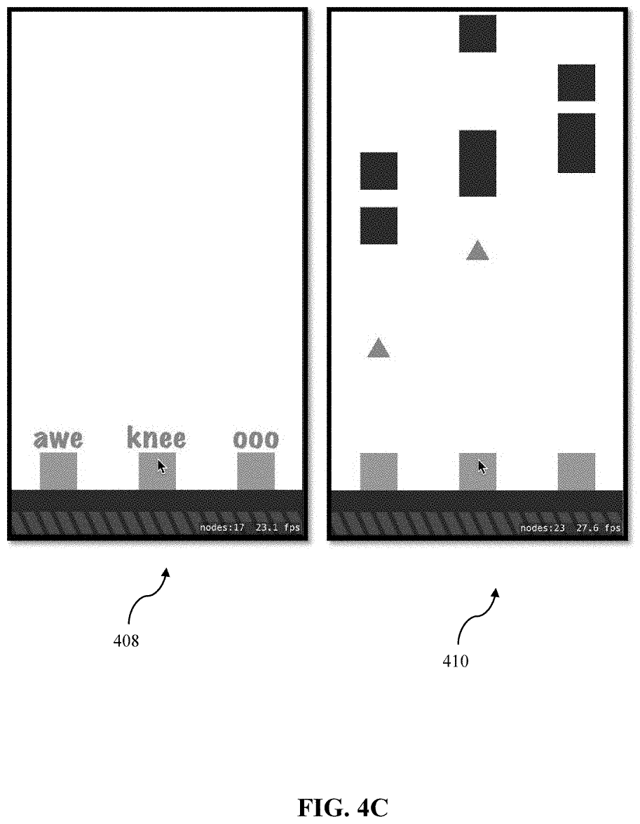

FIG. 1B depicts a system-level block diagram of a device 112 for treatment of snoring or sleep apnea in a patient. The device includes a display 114. Display 114 is the main communication interface for prompting a patient or other user as described above with respect to FIG. 1A. Display 114 can prompt the patient for a vocalization and display an indicator based on the response to that prompt. In embodiments, such as those described in more detail below with respect to FIGS. 3A-4C, display 114 can depict a plurality of targets corresponding to a treatment.

Device 112 also includes an acoustic sensor 118 configured to receive audio input corresponding to a vocalization from the patient or other user. Processor 118 is configured to adjust a position of the indicator on display 114 based on the received audio input at acoustic sensor 116. The vocalization causes the indicator to interact with at least one of the plurality of targets based upon a characteristic of the vocalization. Characteristics that can be used to affect the position of the indicator on display 114 can include, for example, a quantity of time since the vocalization occurred, a duration of the vocalization, a pitch of the vocalization, or a tone of the vocalization.

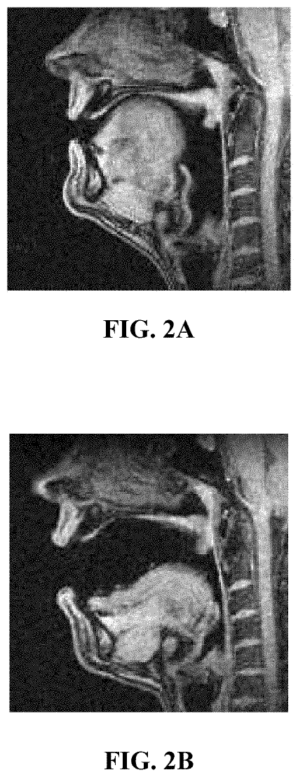

Device 112 can further include transmitter 120, which can send information regarding the patient's compliance with a prescribed vocalization regimen or success rate with the particular tasks or games that are prompted by the device 112 to a health care professional. Transmitter 120 can be a wireless transmitter in some embodiments, while in other embodiments transmitter 120 can be or include or wired transmitter. In embodiments, processor 118 can monitor the interactions between the target and the indicator to determine a level of progress of the patient, which is returned to the heath care professional by transmitter 120. FIGS. 2A and 2B are ultrasonic images depicting the position of the muscles of the upper respiratory system while making /i/ and /a/ vowel sounds, respectively. FIGS. 2A and 2B show how the tongue moves between opposite positions of the upper respiratory system. Some exercise methods as set forth in FIG. 1A may therefore prompt for these two vocalizations, one after the other.

FIGS. 3A-3C are screenshots of an application (“app”) operating on a smartphone, though as previously discussed the app or other software may operate on other devices. The app can be used to train the tongue, nerves, and other muscles associated with snoring and sleep apnea as described above. As shown in FIG. 3A, a display 300 shows an indicator 302. Indicators 302 of FIG. 3A are pillars that extend from the top and bottom of the display 300 to form a gap G therebetween. A user makes sounds according to the targets 306 indicated on display 300. In the embodiment shown in FIG. 3A, targets 306 are positioned to leave a gap G, and targets 306 can move over time. In the embodiment shown in FIG. 3A, as time passes targets 306 moves continuously to the left, indicator 302 moves up or down depending on the audio input from the user. Pitch, tone, volume, duration, or other aspects of a vocalization by the user can constitute audio input from the user that affects the position of indicator 302.

FIG. 3B is a screenshot of an alternate game system in which an indicator 302′ is raised or lowered on a display 300′ to pass through a gap G′ between the targets 306′. As shown in FIG. 3B, targets 306′ of FIG. 3B, unlike targets 306 of FIG. 3A, define jagged walls that run continuously along the top and bottom of display 300′ (with respect to the orientation shown in FIG. 3B). In alternative embodiments, targets 306′ could be smooth walls (rather than jagged), or otherwise define continuous walls or wall segments to direct a patient's vocalizations.

FIG. 3C is a screenshot of an alternate game system in which a display 300″ includes various targets 306″ arranged in columns C1, C2, C3. Each column C1, C2, or C3 corresponds to a different sound. In the embodiment shown in FIG. 3C, column C1 corresponds to the sound “awe,” column C2 corresponds to the sound “knee,” and column C3 corresponds to the sound “do.” As a user makes these sounds, indicators 302″ are produced on display 300″.

In each of the games depicted in FIGS. 3A-3C, the number of times the therapy was successfully completed at the recommended therapeutic level can be measured to provide feedback on the quantity and quality of training of the muscles and nerves associated with prevention of sleep apnea or snoring. Engagement can be measured, including the intensity, frequency, and enjoyment and motivation of the user. Gamification of the training can include controlling objects on a smartphone or other computing device to gain points, badges, progress through levels, and/or other game-based play.

In embodiments, the gamified training programs depicted in FIGS. 3A-3C can also be used to perform diagnostics by analyzing specific vocalizations. These diagnostics can be used to determine how severe a person's snoring or other condition is by the tone, frequency, and/or other acoustic metrics of specific vocalizations. For example, cepstrum analysis can identify potential upper airway exercise therapy that targets either palatal- or tongue-based snoring, based on the diagnosis. The diagnostics can also be used to assess whether a patient will respond to the exercise therapy or to other snoring or sleep apnea treatments. The latter can be used, in embodiments, as a screening tool for other exercise or other therapies.

FIGS. 3A-3C depict three phases of a three-game set. In one embodiment, each of the three games described with respect to FIGS. 3A, 3B, and 3C, respectively, are played for a specific time period, such as five minutes. In embodiments, the order of game play can be randomized, or set according to a health professional's preferences, such that the games are not necessarily played in the order shown in FIGS. 3A, 3B, and 3C.

The training exercise shown in FIG. 3A promotes endurance of the patient's vocalizations. A single phoneme is required of the patient, such as the /i/ sound or /a/ sound. The character on screen 300 moves along a path, and the patient's vocal control is used to move the character up. In embodiments, indicator 302 can move downwards when no sound is made by the patient, to imitate a gravitational effect. In other embodiments, indicator 302 can move downwards at a constant rate when no sound is made by the patient. Endurance is built up because the patient must repeatedly hold the tongue base forward or backward (depending on the phoneme used).

The training exercise shown in FIG. 3B promotes strength by requiring the patient to pulse the tongue backwards and forwards within the upper respiratory system. Two phonemes can be used within the game shown in FIG. 3B. For example in one embodiment the /i/ and /a/ phonemes are used. In another embodiment, /a/ and /u/ phonemes can be used. Indicator 302′ can be raised by producing one of the phonemes and lowered on screen 300′ by producing the other.

The training exercise shown in FIG. 3C promotes coordination by moving the tongue through different zones. Targets 306″ are positioned on the screen 300″ and move downwards with respect to the orientation shown in FIG. 3C. By producing vocalizations corresponding to the columns C1, C2, and C3, targets 306″ can be eliminated. The patient's goal is to “shoot” targets 306″ before they reach the bottom of screen 300″.

FIGS. 4A-4C depict an alternative embodiment of the three games previously described with respect to FIGS. 3A-3C, as well as start screens. FIG. 4A shows an embodiment in which a start screen 400 indicates to a user which vocalization to make for the endurance exercises previously described with respect to FIG. 3A. After display of the start screen 400, a game display is depicted on screen 402. In embodiments, start screen 400 and game screen 402 can be depicted on the same display, with start screen 400 appearing before game screen 402. In alternative embodiments, start screen 400 can be displayed on another screen, or starting instructions can be relayed via audio instructions or some other way.

FIG. 4A further depicts a score 403. In embodiments, a score, a high score, or both, can be depicted on the various screens or displays. This provides a goal for the user to meet or exceed.

FIG. 4B shows start screen 404 and game screen 406 of the strength exercise previously described with respect to FIG. 3B. Likewise, FIG. 4C shows start screen 408 and game screen 410 of the coordination exercise previously described with respect to FIG. 3C.

The gamified training exercises described above provide a specific, structured graphical user interface paired with a prescribed functionality directly related to the graphical user interface's structure that is addressed to and resolves a specifically identified problem in that it provides a simple, non-invasive, and effective way to reduce OSA or snoring. Backed by evidence-based sleep medicine research, the graphical user interface provides a fun smartphone app that uses engaging voice-controlled games build muscle coordination and endurance of the upper airway. Thus, the application has the potential to reduce snoring or sleep apnea without the need for bulky devices or invasive procedures. The app works by guiding the user through a series of games that are easy to understand, yet challenging enough to keep the player engaged. The actions in each game are controlled by the player's vocalizations. Each of these articulations contributes to improving neuromuscular control to reduce apneic events over time. With as little as 15 minutes of gameplay each day, users can significantly improve their airway muscle endurance and reduce apneic events when sleeping.

No other solution for snoring or OSA involves endurance training of the airway through engaging gameplay. There are many devices for OSA, including forced airway masks and surgical implants. All of these devices are invasive and uncomfortable, reducing patient compliance and effectiveness. The gamified application uses the lure and ease of playing fun games on a mobile device without the need for a mask, implant, or other bulky devices.

In addition to providing an innovative therapy mechanism, the app can include a novel diagnostic tool that is able to track the toning and strengthening of the patient's upper airway by tracking changes in the frequency signature of their vocalizations. Currently, diagnosing OSA from patient vocalizations is an active area of research with promising results. The combination of the gamified therapy and a diagnostic tool can not only reduce OSA severity but also indicate which additional therapies would be most effective in further treating OSA for specific patients. This combination would also provide real-time tracking that doesn't require the patient to go into a sleep clinic to see the progress they are making. Giving patients control in their clinical outcomes though positive feedback and showing progress are key elements in retaining patients and ensuring compliance.

Training of the muscles and nerves related to snoring provide benefits over the existing solutions to snoring and sleep apnea. Exercise of the upper airway can result in a 30-50% reduction in Apnea/Hypopnea Index (AHI). Methods for exercising the upper airway include speech therapy, oropharyngeal exercises, orofacial myofunctional exercise, musical instrument exercise, and singing exercise. Each of these can be produced with appropriate gamified exercise therapy as described above, or with alternative exercises that could be incorporated into the gamified app.

Vocalizing two key phonemes, /i/ (as in “he sees these knees”) and /a/ (as in “mama saw claws”) can maximize the range of motion of the hypoglossal muscle and thus exercise the upper airway in the most consistent and efficient manner. The exercises require the patient to complete many different complicated and cumbersome tasks, and without an app to measure and provide feedback, there is not any way for a patient to confirm the patient is performing them correctly short of visiting a clinic or a health care professional. The app provides a considerable improvement, because it provides feedback immediately and without the need for a health care professional regarding whether the patient is properly performing the task (saying “knee” or “awe”) and the physiology ensures proper movement. Furthermore, because the method uses articulations by the patient as an input to control a game, the therapy is fun, engaging, and trackable.

In embodiments, the app facilitates communication of progress by the patient to a health care professional. The health care professional can receive (or request) updates regarding the patient's progress, such as whether the patient uses the program routinely, whether the patient is able to complete the exercises, or how many successful or unsuccessful vocalizations the patient produces (for example, the “high score” information described with respect to FIG. 4A). In embodiments, this information can be transmitted to a health care professional by the device running the app, through any of a number of transmission mechanisms such as via the internet.

Furthermore, by causing the patient to exercise these muscles, rather than stimulating them through use of a device that provides electronic signal, the nervous system associated with these muscles is also trained. This provides advantages over current systems, such as implantable devices that stimulate the genioglossus, because the exercises improve both nervous system and the muscles associated with snoring or sleep apnea.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A method for treatment of snoring or sleep apnea in a patient, the method comprising:
prompting the patient for a vocalization;
displaying an indicator on a display;
displaying a plurality of targets corresponding to a treatment on the display;
receiving an audio input corresponding to the vocalization at an audio sensor; and
adjusting a position of the indicator on the display based on the received audio input, wherein the vocalization causes the indicator to interact with at least one of the plurality of targets based upon a characteristic of the vocalization.

2. The method of claim 1, wherein:
the at least one of the plurality of targets defines a gap; and
causing the indicator to interact with the at least one of the plurality of targets comprises displaying the indicator passing through the gap if the vocalization is in compliance with the treatment.

3. The method of claim 2, wherein the gap is defined by a first target pillar extending from a top of the display and a second target pillar extending from a bottom of the display.

4. The method of claim 2, wherein the gap is defined by a first wall extending continuously along a top of the display and a second wall extending continuously along a bottom of the display.

5. The method of claim 1, wherein the indication corresponding to the vocalization comprises a bullet that interacts with a target of the plurality of targets by removing the target from the display.

6. The method of claim 1, further comprising monitoring the interactions between the target and the indicator to determine a level of progress of the patient.

7. The method of claim 1, further comprising repeating the method with a plurality of prompts.

8. The method of claim 7, wherein for each of the plurality of prompts, if the vocalization produced by the patient matches the prompt, a score of the patient is increased.

9. The method of claim 8, further comprising tracking a high score of the patient.

10. The method of claim 1, further comprising transmitting data corresponding to the interaction of the target and the indicator to a healthcare provider remote from the patient.

11. The method of claim 1, wherein the characteristic of the vocalization is selected from the group consisting of:
a quantity of time since the vocalization occurred,
a duration of the vocalization,
a pitch of the vocalization, and
a tone of the vocalization.

12. A device for treatment of snoring or sleep apnea in a patient, the device comprising:
a display configured to:
prompt the patient for a vocalization,
display an indicator, and
display a plurality of targets corresponding to a treatment,
an acoustic sensor configured to receive audio input corresponding to the vocalization; and
a processor configured to adjust a position of the indicator on the display based on the received audio input, wherein the vocalization causes the indicator to interact with at least one of the plurality of targets based upon a characteristic of the vocalization.

13. The device of claim 12, wherein the processor is further configured to monitor the interactions between the target and the indicator to determine a level of progress of the patient.

14. The device of claim 13, further comprising a transmitter configured to send the level of progress to a healthcare provider.

15. The device of claim 12, wherein the characteristic of the vocalization is selected from the group consisting of:
a quantity of time since the vocalization occurred,
a duration of the vocalization,
a pitch of the vocalization, and
a tone of the vocalization.
